(19) United States
(12) Patent Application Publication
(10) Pub. No.: US 2023/0141622 A1
(43) Pub. Date: May 11, 2023

(54) MINIMALLY INVASIVE NEUROSTIMULATION DEVICE WITH SENSING CAPABILITY

(71) Applicant: Medtronic, Inc., Minneapolis, MN (US)

(72) Inventors: Julia P. Slopsema, Minneapolis, MN (US); Sarah J. Offutt, Golden Valley, MN (US); Katelynn M. Johnson, New Brighton, MN (US)

(21) Appl. No.: 17/983,757

(22) Filed: Nov. 9, 2022

Related U.S. Application Data
(60) Provisional application No. 63/277,448, filed on Nov. 9, 2021.

(51) Int. Cl. A61N 1/36; A61N 1/04

(52) U.S. Cl. CPC A61N 1/36031; A61N 1/0456; A61N 1/0476

(57) ABSTRACT

A leadless neurostimulation device having a header unit having at least one primary electrode that defines an external surface of the device, and a housing that includes a secondary electrode positioned on the same side of device as the primary electrode, a footer coupled to the housing opposite of the header unit, and a controller. The controller configured to operate in a closed-loop to transmit an electrical stimulation signal between the primary electrode to the secondary electrode to provide electrical stimulation therapy to a tibial nerve of a patient, measure a physiologic parameter in response to transmission of the electrical stimulation therapy, and adjust one or more parameters of the electrical stimulation signal based on the measured physiologic parameter.

### CROSS-REFERENCE TO RELATED APPLICATIONS

This application claims the benefit of U.S. Provisional Application Ser. No. 63/277,448, filed Nov. 9, 2021, the disclosure of which is hereby incorporated by reference.

### FIELD

The present application relates to implantable neurostimulation systems, and more specifically to minimally invasive implantable neurostimulation systems with sensing for closed loop control.

### BACKGROUND

Implantable medical devices may be configured to deliver electrical stimulation therapy or monitor physiological signals. Electrical stimulation of nerve tissue, for example, may provide relief for a variety of disorders thereby improving the quality of life for many patients.

Some implantable medical devices (IMDs) may employ electrical leads that carry electrodes. For example, electrodes may be located at a distal portion of an elongate lead. Other examples of electrical leads may be relatively short, having one or more electrodes located along a body of the lead. Such electrical leads are provided separate from the housing or body of the IMD and coupled to the IMD during implantation to provide stimulation via the electrode at a location separated from the housing of the IMD.

Simulation of different nerve branches and clusters have been explored for treating various ailments. One avenue that has shown promising development has been the stimulation of the tibial nerve for the treatment of certain ailments such as incontinence or over-active bladder.

### SUMMARY

Some embodiments of the present disclosure are directed to minimally invasive, leadless neurostimulation devices. Leadless devices do not require the use of a separate lead to deliver stimulation therapy and instead provide a unitary structured device that may be more robust and less invasive than lead-based counterpart devices. The leadless devices further include one or more sensors or electrodes configured to sense nerve activity or muscle activity, and provide closed loop feedback for adjustment of the stimulation therapy regime.

Embodiments of devices disclosed herein may include a housing containing at least one controller and processing circuitry therein configured to deliver neurostimulation therapy, and an attached header unit. The header unit includes one or more primary electrodes that form a portion of the exterior and side of the header unit. The one or more primary electrodes are electrically insulated from other portions of the exterior surface of the neurostimulation device. The housing of the neurostimulation device includes a secondary electrode that operates in conjunction with the one or more primary electrodes to provide electrical simulation therapy or neuro sensing capabilities. The secondary electrode is positioned on the same side of the device as the one or more primary electrodes positioned in the header unit. The device may further include one or more sensors arranged on the housing or footer of the device, the one or more sensors configured to sense stimulated nerve activity. At least one controller (e.g., processor and processing circuitry) configured to receive sensed nerve activity and adjust one or more parameters of the neurostimulation therapy delivered by the primary electrode. The size, shape, and separation distance between the primary electrode(s) and the secondary electrode are discussed and may contribute to more effective and efficient stimulation of the tibial nerve. In some embodiments, the size, shape, and separation distance between the primary electrode(s) and the secondary electrode may be configured to produce an impedance of less than about 2,000 Ohms.

In an embodiment, the disclosure describes a leadless neurostimulation device including a header unit having at least one primary electrode having a contact surface that defines an external surface of the leadless neurostimulation device, and a housing. The housing includes a secondary electrode positioned on the same side of the leadless neurostimulation device as the at least one primary electrode, a footer coupled to the housing opposite of the header unit, and a controller. The controller is configured to operate in a closed-loop to transmit an electrical stimulation signal between the primary electrode to the secondary electrode to provide electrical stimulation therapy to a tibial nerve of a patient, measure a physiologic parameter in response to transmission of the electrical stimulation therapy, and adjust one or more parameters of the electrical stimulation signal based on the measured physiologic parameter.

In another embodiment, the disclosure describes a neurostimulation device that includes a header unit having at least one primary electrode having a contact surface that defines an external surface of the leadless neurostimulation device, and a housing that includes a secondary electrode positioned on the same side of the leadless neurostimulation device as the at least one primary electrode, a footer coupled to the housing opposite of the header unit, a tail lead extending from the footer and comprising one or more sensors, and a controller configured to operate in a closed-loop to: transmit an electrical stimulation signal between the primary electrode to the secondary electrode to provide electrical stimulation therapy to a tibial nerve of a patient, measure a physiologic parameter with the one or more sensors in response to transmission of the electrical stimulation therapy, and adjust one or more parameters of the electrical stimulation signal based on the measured physiologic parameter.

The above summary is not intended to describe each illustrated embodiment or every implementation of the subject matter hereof. The figures and the detailed description that follow more particularly exemplify various embodiments.

### BRIEF DESCRIPTION OF DRAWINGS

The disclosure can be more completely understood in consideration of the following detailed description of various embodiments of the disclosure, in connection with the accompanying drawings, in which:

FIG. 1 is schematic view showing an example leadless neurostimulation device as described herein.

FIGS. 2A-2D are schematic side views of example header units that may be used with the device of FIG. 1.

FIGS. 3A-3E are schematic views of example header units that include a plurality of primary electrodes that may be used with the leadless neurostimulation device of FIG. 1 or with the header unit and electrode arrangements of FIGS. 2A-2D.

FIGS. 4A-4D are schematic side views of example sensor arrangements that may be incorporated into device 10.

FIG. 5 is a schematic side view of a neurostimulation device with a sensor tail.

FIG. 6A is a side view of a patient's leg showing the leadless neurostimulation device of FIG. 1 implanted in a patient's leg near the tibial nerve.

FIG. 6B is a cross-sectional view of a patient's leg showing the leadless neurostimulation device of FIG. 1 implanted near the patient's tibial nerve.

FIG. 7 is a plot showing examples of the minimum threshold level of current needed to observe a tibial nerve response based on a function of return offset in modeling studies using the disclosed leadless neurostimulation devices.

FIGS. 8A and 8B are plots showing modeling results of the effect of secondary electrode radius and the offset of the secondary electrode from the primary electrode in an example header on the stimulation threshold of a model of the tibial nerve located above the Y=0 axis.

FIG. 8C is a plot showing modeling results of stimulation threshold limit for the disclosed device compared to a disc-shaped stimulation device for depth and anterior/posterior relationship.

FIG. 9 is a plot showing the threshold current as a function of stimulation depth for both the 10 mm and 20 mm electrode offsets using a model of the disclosed leadless neurostimulation devices.

FIG. 10 is a comparative plot of models showing the threshold stimulation current as a function of stimulation depth for a comparative disc stimulation device and the disclosed leadless neurostimulation device.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

Embodiments of the neurostimulation devices described herein may be useful for numerous types of neurostimulation therapies, such as for pain control, autonomic nervous system modulation, functional electrical stimulation, tremors, and more. In preferred embodiments, the neurostimulation devices described herein may be useful for both sensing and stimulating one or more nerves to control symptoms of overactive bladder, urge urinary incontinence, urgency frequency, urinary incontinence, stress incontinence, nocturia, painful bladder syndrome, chronic pelvic pain, incontinence, retention, sexual dysfunction, fecal incontinence, intractable constipation, irritable bowel syndrome, inflammatory bowel disease, sexual dysfunction, obesity, gastroparesis, interstitial cystitis, neurogenic bowel or bladder (Parkinson's disease, multiple sclerosis, stroke, spinal cord injury, neuropathy), or other pelvic health conditions. These embodiments may also be useful for both sensing and stimulating one or more peripheral nerves to control pain in one or more areas of the body, such as a foot, ankle, leg, groin, shoulder, arm, wrist, or the back, for example. In one example, embodiments of the disclosed neurostimulation devices may be used to both sense and stimulate a tibial nerve of a patient.

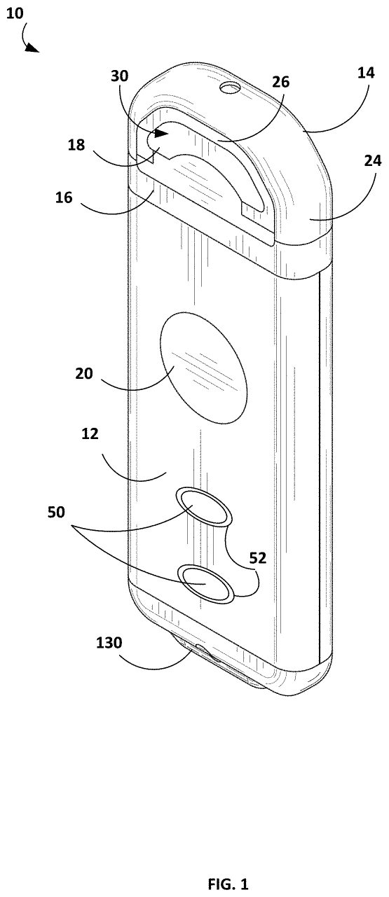

FIG. 1 is schematic view showing an example leadless neurostimulation device 10. Leadless neurostimulation device 10 includes a housing 12 containing components therein configured for delivering neurostimulation therapy, a header unit 14 that includes one or more primary electrodes 18, and a mounting plate 16 that couples housing 12 to header unit 14. Header unit 14 includes at least one primary electrode 18 that forms part of an exterior surface of header unit 14. Housing 12 includes a secondary electrode 20 that forms part of an exterior surface of housing 12 and is positioned on the same side of device 10 as primary electrode 18. In an alternate embodiment not depicted, primary electrode 18 and secondary electrode 20 may be arranged on opposite sides of device 10. Device 10 further includes one or more sensors 50 positioned along housing 12.

Primary electrode 18 and secondary electrode 20 operate in conjunction with one another, e.g., via a controller, to provide stimulation therapy to a target treatment site (e.g., a tibial nerve). Secondary electrode 20 may also be referred to as a case electrode, can electrode, or reference electrode. In an embodiment, primary electrode 18 may comprise a cathode and secondary electrode 20 may comprise an anode. In some embodiments, primary and secondary electrodes 18 and 20 may be characterized as a bipolar system or pair of electrodes.

The terms “primary” and “secondary” are used to differentiate two or more electrodes that are configured to transmit an electrical signal therebetween. The terms are not used to imply a hierarchy among the electrodes, positive and negative terminal, a total number of electrodes, or a directionality by which a signal is transmitted between the electrodes. In some embodiments, electrode 18 may be configured as the secondary electrode and electrode 20 configured as the primary electrode. In some embodiments, electrodes 18 and 20 may be switched between primary and secondary as desired.

One or more sensors 50 may be operated by a controller and processing circuitry within device 10 for sensing a physiologic parameter indicative of nerve or muscle activity measured in response to stimulation of the targeted nerve by primary electrode 18. For example, sensors 50 may be configured to sense nerve activity based on measuring or monitoring an electromyogram (EMG) signal, e.g., a neuromuscular response, evoked compound action potential (ECAP), e.g., nerve capture but without the neuromuscular response, or combinations thereof, to provide real-time indication regarding the effectiveness of the stimulation therapy. Such signals may include M-wave, H-wave, F-wave, and combinations thereof. The EMG signal or ECAP response may occur shortly after the delivered electrical stimulation therapy, e.g., less than a millisecond (ms) allowing for a direct correlation between applied stimulation and measured response.

A response signal obtained by sensors 50 (or selected electrode/sensor combination) may be used by the controller and processing circuitry of device 10 to control and adjust the stimulation parameters of the stimulation therapy being delivered by primary electrode 18. The information may be used in a closed-loop feedback system to optimize or adjust one or more of the stimulation parameters for better or more effective therapy. Such adjustments to the simulation therapy may include a change in an amplitude or pulse width of the stimulation signal, change in the selected primary electrode 18 or electrode pair (e.g., if a plurality of primary electrodes are present) to ensure better capture of the targeted nerve, change in frequency, change in sensor or stimulation configurations, and the like.

Header unit 14 includes outer housing 24, primary electrode 18, and dielectric mount 26. Outer housing 24 is coupled to mounting plate 16 and may define a partially recessed cavity that receives dielectric mount 26 and primary electrode 18. Outer housing 24 and mounting plate 16 may be made of metal or metal alloy (e.g., titanium or titanium alloy) to allow for easy coupling there between (e.g., laser welding) as well as allow for the coupling of mounting plate 16 to housing 12. Additionally, or alternatively, outer housing 24 or mounting plate 16 may be composed of a ceramic material, or non-conductive plastic material (e.g., polypropylene) including appropriate mechanisms (e.g., metal inserts) for coupling outer housing 24 to mounting plate 16.

In some embodiments, the seam between mounting plate 16 and outer housing 24 may form at least a partial hermetic seal. In an alternate embodiment not depicted, header unit 14 may be configured so as to be coupled directly to housing 12, without the need for a separate mounting plate element.

Primary electrode 18 defines exterior contact surface 30 configured to be brought into direct contact with tissue of the patient. Contact surface 30 may also form a portion of a side of header unit 14, which is preferably on the same side of device 10 as secondary electrode 20.

The exterior perimeter of contact surface 30 is at least partially bordered by dielectric mount 26, which may also form a portion of the exterior surface of header unit 14 absent of the dielectric coating disclosed below. Dielectric mount 26 electrically insulates and physically separates primary electrode 18 from outer housing 24, mounting plate 16, and other portions of device 10. Additionally, or alternatively, dielectric mount 26 may be molded around primary electrode 18 using silicone or liquid silicone rubber LSR, for example, to help physically retain primary electrode 18 within header unit 14 of device 10. In some embodiments, dielectric mount 26 may be formed integrally with outer housing 24 provided the components are formed of a non-conductive material.

Outer housing 24 may form the majority of the body of header unit 14. In particular, outer housing 24 forms the side of header unit 14 opposite of contract surface 30, the perimeter edges (apart from the contact surface provided with mounting plate 16), and a portion of the same side of header unit 14 as contact surface 30 of primary electrode 18. In some embodiments, outer housing 24 may have a rounded, semi-circular, or D-shaped perimeter edge that provides a relatively smooth surface without any abrupt or sharp edges or lines than may present an irritation to the patient after implantation. In some embodiments, outer housing 24 is configured to receive and form a partial shell around dielectric mount 26. As such, outer housing 24 may define a concave interior surface (not shown) that receives a portion of dielectric mount 26. Dielectric mount 26 may be secured to outer housing 26 using a suitable adhesive material (e.g., non-conductive medical adhesive, epoxy volcanized silicone, or the like).

Primary electrode 18 may be of suitable shape to provide electrical stimulation to the tibial nerve through the fascia layer of a patient. In some embodiments, contact surface 30 of primary electrode may be substantially flat (e.g., flat or nearly flat) as shown in FIG. 1. Alternatively, primary electrode 18 may define a curved surface (e.g., a semi-cylindrical shape or other 2D or 3D curved plane) that helps primary electrode 18 follow the curvatures of the fascia layer of a patient when implanted to provide better contact and focusing of the electrical signal directed to the tibial nerve. The curved surface may extend over the entirety of contact surface 30, or only over a portion of the surface. Additionally, or alternatively, the curvature may be confined to only contact surface 30 of primary electrode 18, or may extend over other portions of device 10 such as other parts of header unit 14, mounting plate 16, or housing 12. By including the curvature over other portions of device 10, the device may provide a more ergonomic fit when implanted while also helping to direct the stimulation signal to the tibial nerve.

In some embodiments, contact surface 30 of primary electrode 18 may also protrude from the plane defined by housing 12. Such a protrusion may help apply additional pressure to the fascia of the patient and help guide the electrical stimulation signal deeper into the tissue of the patient. Primary electrode 18 may also define one or more interlocking features, carveouts, recesses, or other structures that reduce the overall volume of primary electrode 18 without interfering with contact surface area 30. The reduced volume and interlocking features may also help reduce manufacturing costs as well as help fix primary electrode 18 relative to dielectric mount 26.

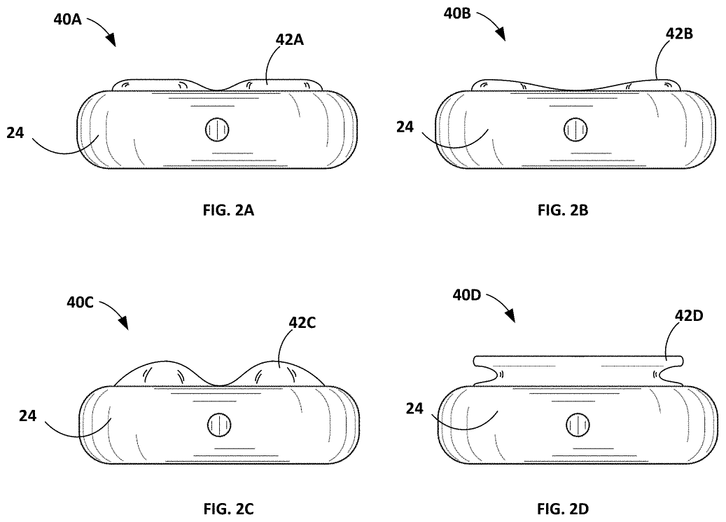

FIGS. 2A-2D are schematic side views (top-down) of example header units 40A-40D that may be used with device 10 of FIG. 1. Each header unit 40A-40D includes one or more primary electrodes 42A-42D that may be curved, protrude away from the plane defined by housing 12, or both. The curvatures shown in FIGS. 2A-2C generally curve relative to the centerline defined by device 10 (e.g., into the page in FIGS. 2A-2C through the center of the device) to help focus the electrical stimulation, sensing, or both to a line substantially parallel (e.g., parallel or nearly parallel) to the center line of device 10. Additionally, or alternatively, by protruding primary electrodes 42A-42D, the electrode may lie in closer proximity to the tibial nerve compared to secondary electrode 20 which can help guide or steer the electrical stimulation to the nerve allowing for deeper nerve stimulation (e.g., stimulation of tibial nerve with deep or anterior/posterior tracks).

The material of primary electrode 18 (or sensors 50 described below) may depend on the type of signals to be measured and the electrical stimulation therapy to be delivered. In some examples, primary electrode 18 or sensors 50 of device 10 may be made from, but are not limited to titanium, titanium alloy, platinum iridium, or the like. In some embodiments, at least contact surface 30 is formed of platinum iridium, which provides low impedance to bodily tissue. The body of primary electrode 18 may be made of the same or different material than contact surface 30. For example, primary electrode 18 may be formed of titanium with contact surface 30 formed of platinum iridium. Using platinum iridium or titanium may be beneficial in reducing or eliminating the potential for charge buildup on the external surface of device 10 during operation. Alternatively, primary electrode 18 may be formed out of titanium (e.g., commercially pure titanium) or a titanium alloy, which may provide cost or assembly benefits in the construction of device 10 compared to forming primary electrode out of platinum iridium. In some examples, forming rounded edges on the electrodes or sensors may also help avoid edge defects.

Header unit 14 is coupled to mounting plate 16 and likewise mounting plate 16 is coupled to housing 12. Housing 12 includes secondary electrode 20. In some embodiments, secondary electrode 20 may be defined by an area of the body of housing 12. For example, housing 12 may be formed of a metallic material (e.g., titanium) and electrically coupled to the processing circuitry of leadless neurostimulation device 10 so that housing 12 forms part of the electrical circuit. The outer surface of housing 12, including portions of mounting plate 16, header unit 14, and footer 54, may be coated with a dielectric material apart from the surface area that defines secondary electrode 20, primary electrode 18, and sensors 50. The dielectric material may at least partially encapsulate device 10 such that the boundary created by the dielectric material in turn defines the area of secondary electrode 20, contact surface 30, or both.

Alternatively, housing 12 may include an aperture through the body of housing 12, and secondary electrode 20 may be positioned within the aperture. In such examples, secondary electrode 20 would be a standalone component separate and independent of housing 12 such as secondary electrode 20 is electrically isolated from housing 12. While this configuration is recognized as a possible arrangement, the below description assumes that housing 12 forms secondary electrode 20 such that the two components are one and the same with secondary electrode 20 being defined based on the application of the dielectric coating to the exterior surface of housing 12.

The dielectric coating may be applied using any suitable technique. In some such examples, the areas defining contact surface 30 and secondary electrode 20 (and other areas of device 10 not intending to receive the application of the dielectric coating) may be masked with a suitable material such as tape. Leadless neurostimulation device 10 may be then coated using vapor deposition, dip coating, spray coating of similar technique with an adherent dielectric material followed by subsequent removal of the mask material to expose the surfaces of contact surface 30 and secondary electrode 20.

Suitable dielectric materials for coating leadless neurostimulation device 10 may include, but are not limited to, parylene, LSR, or silicone. Additionally, or alternatively, the outer surface of neurostimulation device 10 or portions thereof, may include a surface treatment such as an anodization treatment to modify portions of the surface to make the surface non-conductive. For example, portions of housing 12, outer housing 24, or both, if made of metal (e.g., titanium) may be treated through anodization to make select surfaces non-electrically conductive. In such examples, for purposes of this disclosure the exterior surface of the components may still be characterized as being metal (e.g., titanium) although the component has received such surface treatment.

In preferred examples, the outer surface of leadless neurostimulation device 10 may be formed primarily of parylene. Formation of the desired electrode profiles may utilize dielectric blocking methods (e.g., use of a masking material during manufacture) or dielectric removal methods (e.g., removal via laser or soda blast) without damaging the dielectric coating.

In some embodiments, the dielectric coating may also contribute to creating a hermetic seal around leadless neurostimulation device 10. The general configuration of attaching header unit 14 and housing 12 respectively to mounting plate 16 may also produce a hermetic seal within device 10. Coating device 10 with a dielectric material possessing sealing properties such as parylene, LSR, or silicone may either provide additional robustness to the hermetic seal of device. Providing leadless neurostimulation device 10 in a hermetically sealed form may contribute to the device's long-term functionality thereby providing advantages over other non-hermetically sealed devices.

The controller, processing circuitry, and other components of neurostimulation device 10 are contained within housing 12. Examples of such components may include one or more electronic circuits for delivering electrical stimulation therapy, telemetry hardware, power supply, memory, processor(s). The one or more processors (e.g., controller) may be used to control one or more parameters of the stimulation therapy and receive sensor information from the one or more sensors 50. As discussed further below, the received data from the sensors may be used in a controlled-loop feedback system to adjust a parameter of the stimulation therapy to improve or optimize the therapy regime. Housing 12 can also include communication circuitry disposed therein for receiving programming communication from an external programmer (e.g., external programing device), or providing feedback to a programmer or other external devices.

In one example, housing 12 can include an energy source enclosed therein, e.g., a rechargeable or non-rechargeable battery. In another example, leadless neurostimulator 10 can also be configured to receive energy signals from an external device and transduce the received energy signals into electrical power that is used to recharge a battery of the device, an energy source e.g., a battery, processing circuitry, and other necessary components enclosed therein. In some embodiments, device 10 can be configured to receive energy signals from an external device and transduce the received energy signals into electrical power that is used to recharge a battery of device 10. Additionally, or alternatively device 10 may include a non-rechargeable primary cell battery.

In some embodiments, housing 12 of leadless neurostimulation device 10, and its various processing components may be substantially similar to the housing portion of the InterStim Micro Neurostimulator available from Medtronic. The InterStim Micro Neurostimulator may be modified to receive header unit 14 described herein along with modifications to provide secondary electrode 20 and sensors 50. The total volume of neurostimulation device 10 may be relatively small as well: 0.5 cubic centimeters (cc) to about 6 cc, about 1.5 cc to about 3.5 cc, or about 2 cc to about 3 cc.

The size, shape, and physical separation distance between primary electrode 18 and secondary electrode 20 can affect the functionality and effectiveness of leadless neurostimulation device. In some embodiments, primary electrode 18 may define a contact surface area of about 5 mm² to about 90 mm². In preferred embodiments that include only a single primary electrode 18, the contact surface area may be greater than about 10 mm², greater than about 15 mm², greater than about 18 mm², greater than about 20 mm², less than 35 mm², less than 30 mm², and less than 25 mm². In embodiments, primary electrode 18 may define a contact surface area between about 12 mm² and 22 mm². Secondary electrode 20 may define a contact surface area of about 40 mm² to about 120 mm². In embodiments, secondary electrode 20 may define a contact surface area between about 60 mm² and 71 mm². However, devices having larger sized secondary electrodes may increase the minimal current needed to create a therapeutic response. The separation distance between primary electrode 18 and secondary electrode 20 may be about 5 mm to about 20 mm.

In some embodiments, the size, shape, and physical separation distance between primary electrode 18 and secondary electrode 20 may be configured to produce an impedance of less than 2,000 ohms (e.g., between about 100 ohms and 1,000 ohms). In other embodiments, the size, shape, or physical separation distance between electrodes may be configured to produce an impedance greater than 2,000 ohms. Additionally, or alternatively, primary and secondary electrodes 18 and 20 may be arranged in a non-concentric arrangement such that one electrode does not substantially encircle the other.

In some embodiments, one or more of primary and secondary electrodes 18 and 20 may be configured to operate in one or more modes including one or more sensing modes where, for example, the electrode is used to detect measurable feedback from the tibial nerve (e.g., sensed activity or the nerve prior to or after stimulation) or sense the relative location of the tibial nerve to optimize stimulation and a delivery mode where the electrode delivers stimulation therapy to the tibial nerve. The processing circuitry may select one or more optimal electrodes based on proximity to the tibial nerve for the delivery of stimulation therapy so as to steer the stimulation field. Additionally, or alternatively, in a sensing mode, one or more of primary and secondary electrodes 18 and 20 may be configured to monitor the activity of the tibial nerve or adjacent tissue prior to or during the delivery of simulation therapy to determine if sufficient therapy has been delivered. The sensory mode may be actuated by processing circuitry contained in the body of housing 12.

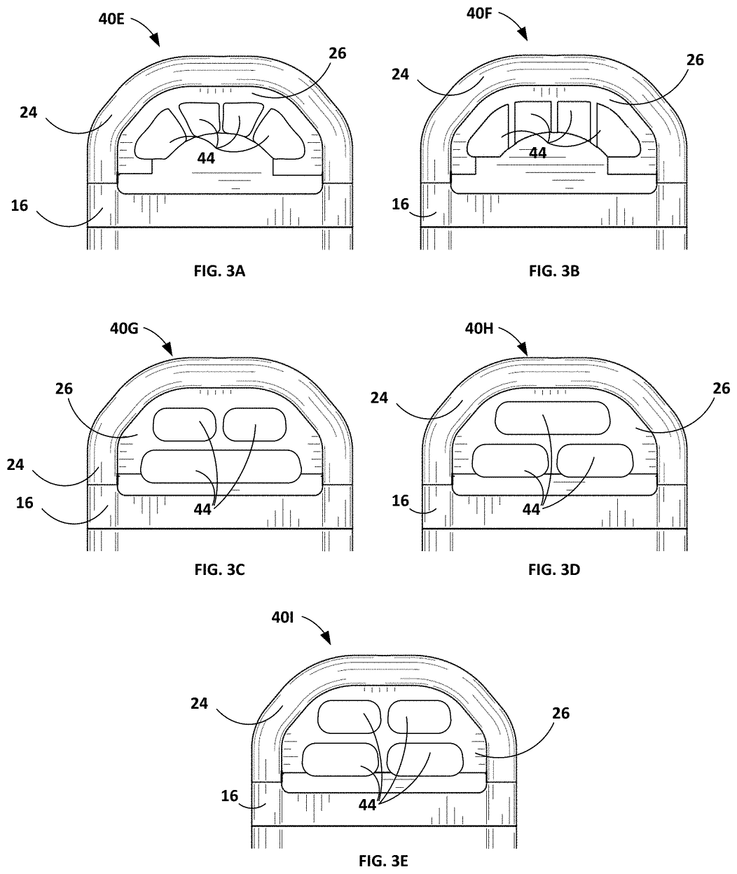

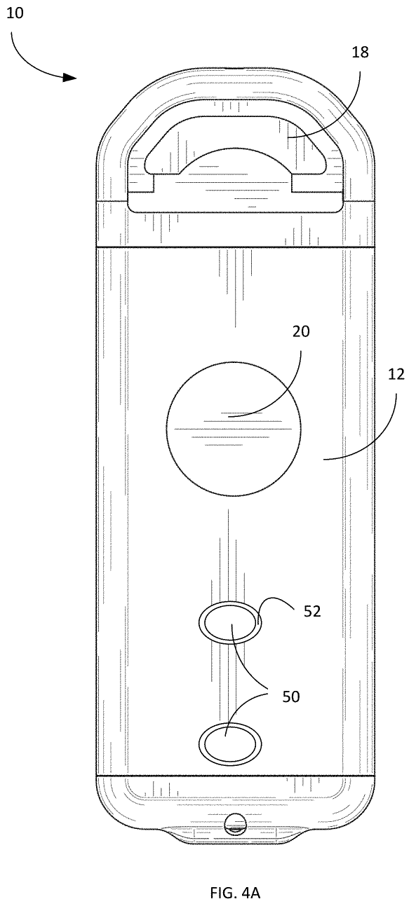

In some embodiments, header unit 14 may include a plurality of primary electrodes 18. FIGS. 3A-3E are schematic views or example header units 40E-401 that each include a plurality of primary electrodes 44. In some embodiments primary electrodes 44 may be similarly sized and shaped or include a collection of differently shaped and sized electrodes. Having relatively rounded edges along primary electrodes 44 can help reduce charge buildup defects.

The inclusion of more than one primary electrode 44 in device 10 may increase functionality and precision of device 10. For example, one or more of primary electrodes 44 may be configured to operate in one or more modes and allow for selection of an appropriate electrode 44 via appropriate switching circuitry to deliver therapy. Having multiple primary electrodes 44 provides the option of selecting a particular electrode or electrode pair that is best positioned for the delivery of stimulation therapy. This improved stimulation targeting could limit possible side effects from stimulating unintended areas. Additionally, improved targeting through the use of sensory or neural feedback using for example sensors 50 as discussed further below may also help improve stimulation therapy by allowing for customizable stimulation parameters such as modification to the stimulation signal (e.g., adjustment to the stimulation voltage, amplitude, duration, and the like) or provide options for unique therapy applications (e.g., providing stimulation to two sides of the nerve simultaneously or interpretation of different types of sensory signals) using one or multiple wave forms. For example, in response to sensory data received from one or more sensors 50, the processing circuitry may select one or more optimally placed primary electrodes 44 based on proximity to the tibial nerve for the delivery of stimulation therapy to help steer the stimulation field.

In some examples, primary electrodes 44 may be used for both stimulation and sensing purposes. In such arrangements device 10 may include a controller and processing circuitry configured to selectively switch between stimulation and sensing operational modes using preselected or user selected electrode pair combination. For example, one of primary electrodes 44 may be used for delivering stimulation therapy to a target site in conjunction with secondary electrode 20, while the same or a different primary electrode 44 may be used in a sensing operational mode to determine if sufficient electrical stimulation therapy has been delivered to the target treatment site. In such examples, the primary electrode 44 being used for sensing purposes may be used in conjunction with one of the other primary electrodes 44 or secondary electrode 20 to complete the sensing circuit. Switching circuitry within device 10 may alternate between stimulation and sensing modes to allow for use of the selected electrode pair combination in the desired operational mode, and may also alternate between selections of electrodes.

In some examples, one or more of primary electrodes 44 may be configured to operate in one or more modes including one or more sensing modes where, for example, the electrode is used to detect measurable feedback from the tibial nerve (e.g., sensed activity or the nerve prior to or after stimulation) or sense the relative location of the tibial nerve to optimize stimulation and a delivery mode where the electrode delivers stimulation therapy to the tibial nerve. The processing circuitry may select one or more optimal primary electrodes 44 based on proximity to the tibial nerve for the delivery of stimulation therapy so as to steer the stimulation field. Additionally, or alternatively, in a sensing mode, one or more of primary electrodes 44 may be configured to monitor the activity of the tibial nerve or adjacent tissue prior to or during the delivery of simulation therapy to determine if sufficient therapy has been delivered. The sensory mode may be actuated by processing circuitry contained in the body of housing 12.

In some examples, one or more of the primary electrodes 44, not being used in the active stimulation mode, may be used with one or more of sensors 50 as an independent circuit to the stimulation operation. In such examples, device 10 may be configured to both deliver stimulation therapy and sense information in response to the delivery of stimulation therapy based on the two different electrical pathways. The stimulation and sensing operations may be performed simultaneously to provide real-time feedback, or may be performed in sequence where device 10 alternates between stimulation and sensing operational modes.

In some examples, selection of the desired primary electrode 44, secondary electrode 20, and sensor 50 pair combination for a desired operational mode (e.g., stimulation or sensing modes) may be done by a clinician or a programmer through a separate programming device containing a user interface. For example, a clinician or programmer can use a separate programming device (not shown) that allows the clinician to select which electrode-pair combination in device 10 that should be designated as the stimulation electrodes, and which electrode or sensor pair combination should be designated for the sensing configuration. The programming device may further provide the clinician or programmer the option of having the stimulation and sensing modes performed either simultaneously, or in alternating sequence, depending on which electrode pairs are selected for the different operational modes and whether simultaneous operation is permissible with the selected pairs.

In some examples, the programming device may also have a calibration mode that provides feedback information to the programmer or clinician to help optimize which electrode or sensor pair is best positioned for performing the different operational modes. For example, during such calibration, device 10 may provide a test stimulation using a given electrode combination and obtain sensor information using different electrode/sensor pairs in response to the electrical stimulation. This sensor information may be displayed to the clinician or programmer through the programming device so that the user can determine which electrode pair combination is best positioned for sensing a particular type of signal in response to the stimulation therapy. The user may then be given the option of selecting that particular electrode/sensor pair to conduct the sensory measurements in the closed loop feedback system.

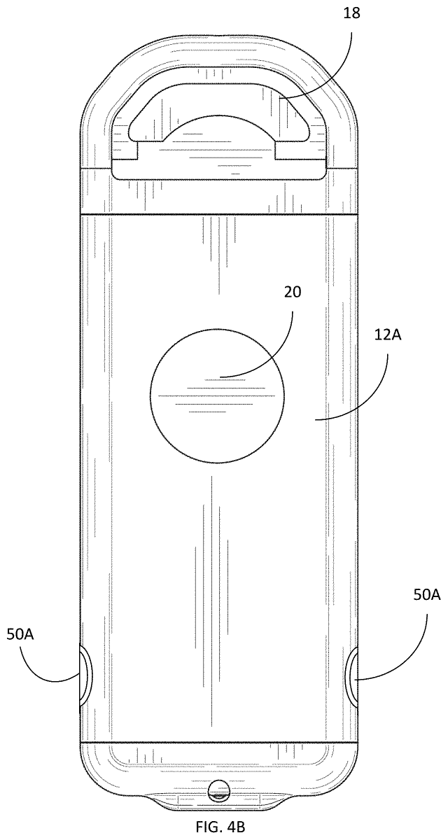

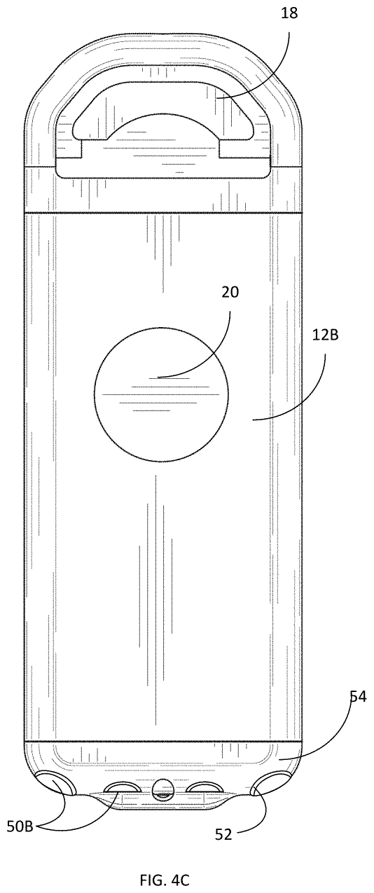

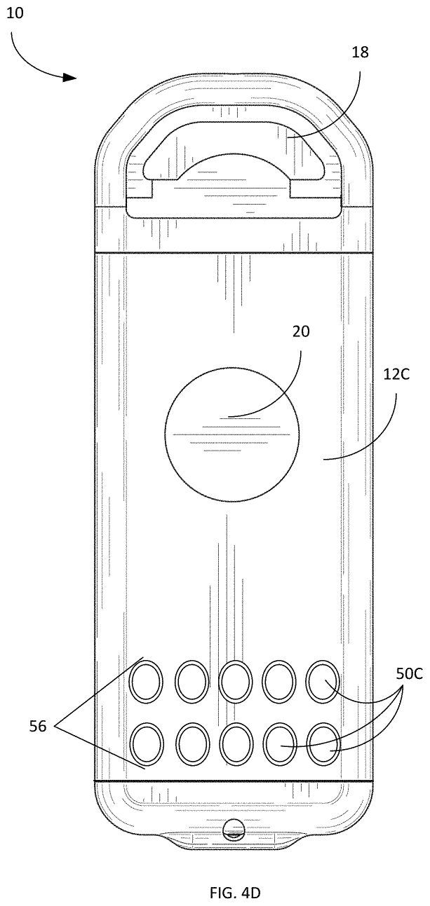

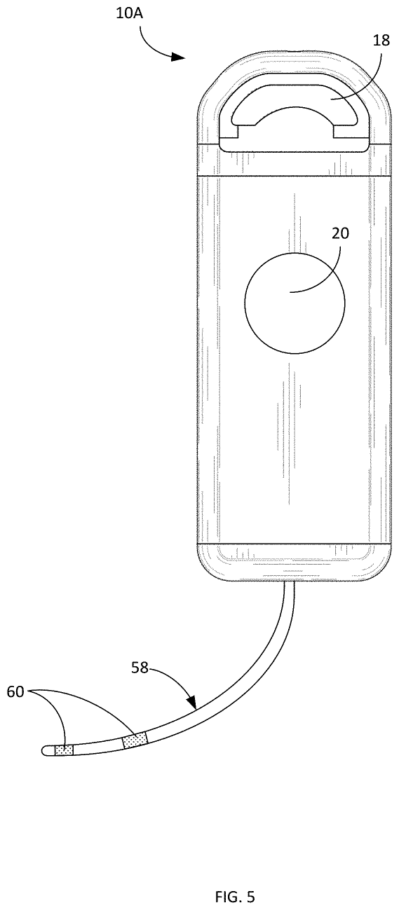

FIGS. 4A-4D show side views of device 10 and examples of possible combinations and placements for the disclosed sensors. As shown in FIG. 4A, housing 12 may include one or more sensors 50 contained thereon for sensing various parameters of nerve traffic or muscle activity. For example, the electrode sensors 50 may be configured to sense nerve activity based on, or in response to, the stimulation provided by primary electrode 18. Through such sensing, sensors 50 may measure nerve activity (e.g., activity of the tibial nerve) to provide real-time indication regarding the effectiveness of the stimulation therapy that may be used by the controller, including processing circuitry, to adjust one or more parameters associated with the stimulation therapy.

In embodiments, sensors 50 are generally described in this disclosure as being present in quantities of two or more and operating independent of the stimulation electrodes (e.g., primary and secondary electrodes 18 and 20). Having sensors 50 operate independent of the stimulation electrodes allows sensors 50 to be operated simultaneously with the stimulation electrodes. This in turn allows for sensing to be coordinated with stimulation therapy to assess and determine whether the parameters of the stimulation therapy need to be adjusted, in real time, to optimize the stimulation. In an alternative embodiment, device 10 may include a single sensor 50 configured to function in conjunction with stimulation electrodes such that device can both stimulate and sense nerve traffic or muscle response at the same time to provide real time analysis of the stimulation therapy parameters.

In other examples, as indicated above, one or more of sensors 50 may be provided by one of the stimulation electrodes. For example, device 10 may include a single sensor 50 that operates with secondary electrode 20 as the reference electrode, and/or one of primary electrodes 44 may be operated in a sensing capacity. In such examples, device 10 may include switching circuitry to oscillate between stimulation and sensing modes. In the stimulation mode, primary electrode 18 and secondary electrode 20 may function as described above to provide stimulation therapy. Device 10 may then switch to a sensing mode where sensor 50 and secondary electrode 20, or other components, operate to determine muscle or nerve activity in response to the stimulation therapy. Device 10 would then oscillate between stimulation and sensing modes to optimize stimulation therapy, while there would be a partial delay between delivering stimulation therapy and sensing nerve or muscle fiber response activity. In such embodiments, device 10 may conserve space by having a reduced overall volume due to the reduced number of electrodes needed to be included with device 10. In some examples device 10 may include up to four sensors/electrodes distributed in a combination primary electrode 18 and sensor 50 in any desirable combination (e.g., one primary electrode 18 and three sensors 50, two primary electrodes 18 and to sensors 50, or the like).

In some examples, sensors 50 may be exposed through one or more apertures within housing 12. In embodiments, housing 12 may be used as the conductive material forming secondary electrode 12, and sensors 50 may be electrically isolated from housing 12 by dielectric material 52 that electrically isolates sensors 50 from one another and from the substrate material of housing 12. Dielectric material 52 may be the same material as dielectric mount 26 or constructed from another suitable material. Sensors 50 may include any suitable type of electrode sensor device that is electrically coupled to electronic circuitry of device 10 by, for example, conductive traces or the like. Further, sensors 50 may be constructed of any suitable materials including the same materials described above with respect to primary electrode 18.

Sensors 50 or the electrode/sensor pair selected for operation in the sensory mode, may be configured to measure various signals designed to assess the efficacy of stimulation therapy provided by the stimulation electrodes or to avoid side effects detected by the sensing electrodes. Useful signals may include monitoring an electromyogram (EMG) signal or evoked compound action potential (ECAP), or combinations thereof. The signals may be obtained and analyzed by processing circuitry of device 10 to determine, for example, whether the proper nerve(s) is being stimulated by the stimulation therapy or the proper muscle response is evoked, whether the secondary or unintended nerve or muscle fibers are being stimulated by the stimulation therapy, whether the amplitude of the stimulation electrodes is sufficient to trigger a threshold therapeutic response or to evoke a response to the correct nerve fibers, whether the stimulation therapy is excessively high for the desired therapeutic effect, or the like.

For example, some applications of electrical stimulation can trigger an electromyography (EMG) signal. A measured EMG, within a specific time window related to nerve conduction up and down the leg at specific locations (e.g., great toe, lesser toes), may indicate that the medical device adequately stimulated the nerve. The EMG signal may be used to determine if the amplitude of the stimulation therapy is sufficient to trigger a desired response. As an example, H-wave or F-wave responses analyzed as part of the EMG signal acquired for tibial nerve stimulation may be used to as an indicator that motor neurons in the sacral plexus or spinal cord have been sufficiently activated. Additionally, the strength of the EMG signal response over time may indicate the plasticity that repeated stimulation may induce and thereby provide an indication relating to overall effectiveness of a given stimulation therapy. Device 10 may operate in a closed loop system, configured to alter the parameters of the stimulation therapy, e.g., stimulation level, pulse width, pulse pattern or other parameters dependent on the amplitude or other characteristics of the EMG signal. In some embodiments, inspection of the EMG waveform, e.g., the peak arrival time or a zero-crossing arrival time, can assist in determining if the proper nerve tissue is being stimulated versus non-targeted fiber tissue. For example, a latency in the arrival time may indicate that nearby muscle or other muscle innervated by the target nerve is being stimulated, rather than the target nerve itself. Such information can be used by device 10 to adjust one or more sensing or stimulation settings to improve device operation.

Another example of the electrical signal generated by the patient in response to an electrical stimulation greater than or equal to the sensory threshold is an evoked compound action potential (ECAP), which is synchronous firing of a population of neurons which occurs in response to the application of a stimulus including, in some cases, an electrical stimulus by device 10. Inspection of the ECAP waveform, e.g., the peak arrival time or a zero-crossing arrival time, can assist in determining if the proper nerve tissue is being stimulated versus non-targeted fiber tissue. Further, threshold detection of the signals can indicate a minimum amplitude of electrical stimulation is being provided. However, measurement of the ECAP may be useful to determine if a minimum threshold electrical stimulation has been delivered, and may also be useful in determining number of fibers stimulated, a percentage of the fibers being captured within a targeted group, or the type of fibers being stimulated (e.g., alpha fibers, beta fibers, c-fibers, and so forth).

The sensory data collected by the selected sensors/electrode pair (e.g., detection of EMG signal or ECAP) may be used by the controller in a closed-loop feedback system configured to monitor and optimize the electrical stimulation therapy provided by device 10. For example, the collected sensor data may be used to determine whether a minimum or maximum desired electrical stimulation therapy is being delivered to the target treatment site, whether an appropriate target nerve fiber is being captured by the electrical stimulation, may help mitigate device migration concerns or reduce the amplitude of stimulation provided if an otherwise non-optimally placed electrode were selected for the delivery of electrical stimulation, or the like. Additionally, sensory data can help to “steer” where the stimulation therapy is delivered after device 10 has been implanted thereby reducing the time needed during implantation otherwise used to assess proper placement.

Whether sensors 50 are configured to operate in an EMG, ECAP, simultaneous EMG/ECAP, or other sensor mode may be chosen by the controller and processing circuitry or by the clinician (e.g., through an external programming device) without modification to the hardware used in device 10. However, whether sensors 50 are intended to work in one sensory mode or another may affect the general orientation and placement of sensors 50 on device 10. For example, in an ECAP sensory mode, sensors should be oriented on device 10 so as to be aligned along and parallel to a length of the tibial nerve, or other target nerve or muscle tissue, when device 10 is implanted. Placing sensors 50 in such a linear and parallel fashion allows for the ECAP signal to be measured based on the readings at proximal and distal sections of the target nerve or muscle fiber. Thus, in some embodiments, it may be beneficial for sensors 50, and primary and secondary electrodes 18 and 10, to be arranged in a substantially linear (e.g., linear or nearly linear) fashion along a longitudinal length or axis of device 10 as depicted in FIGS. 1 and 4A for example. Such an arrangement may increase the overall capabilities of device 10. The general alignment of sensors 50 may be less important for detecting EMG signals, thus allowing greater options for placement locations of sensors 50.

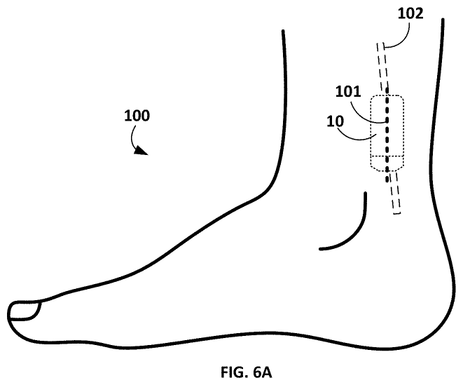

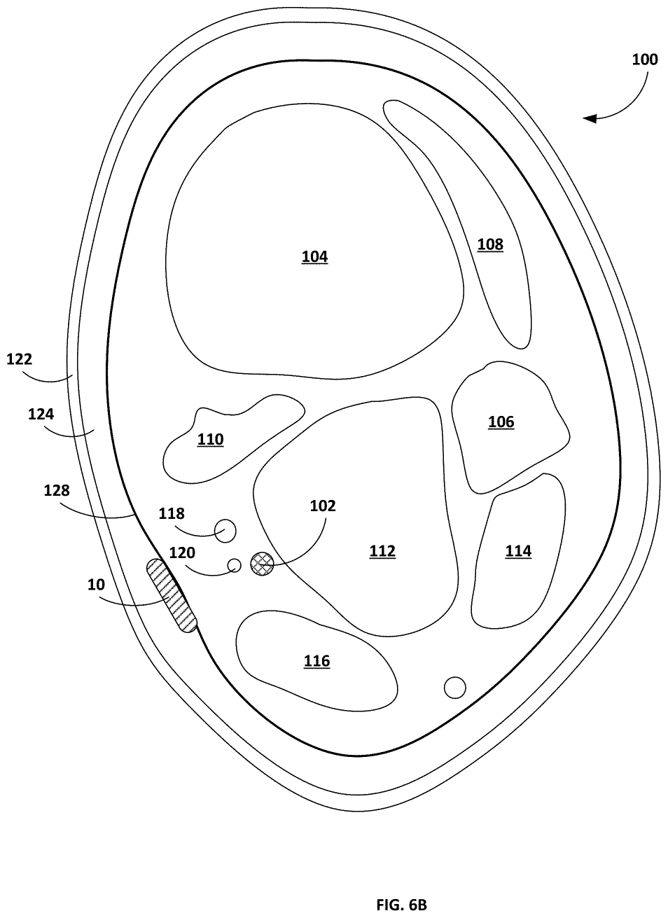

Sensors 50 may be positioned along device 10 so that sensors are sufficiently separated from primary electrode 18 to reduce the amount of signal interference/artifact generated during electrical stimulation. In some examples, sensors 50 and primary electrode 18 may be generally positioned at opposite ends of device 10, which may reduce the amount of signal interference generated by the stimulation therapy. Additionally, because sensors 50 are designed to assess responsive nerve or muscle activity due to the delivery of stimulation therapy, placing sensors 50 along the lower edge of device 10 opposite of primary electrode 18, sensors 50 will be positioned closer to the patient's knee (see FIG. 6A) compared to primary electrode 18 thus capturing responsive traffic generated by primary electrode 18. Additionally, it may be beneficial to maximize the separation distance between sensors 50 and primary electrode 18 and secondary electrode 20 to reduce the amount of noise generated by the stimulation therapy. In some examples, sensors 50 may be separated from primary electrode 18 (e.g., edge-to-edge) by a distance of at least 3 mm away, more preferably at least 5 mm of edge-to-edge separation.

While the example in FIG. 4A shows only two sensors 50, aligned linearly with primary and secondary electrodes 18 and 20 and being on the same surface (e.g., within the same plane) as secondary electrode 20, in another example, sensors 50 may be positioned at other positions along device 10, such as positioned along one or more perimeter surfaces of device 10 opposite of secondary electrode 20 (e.g., on the backside of device 10). For example, FIG. 4B shows a side view of housing 12A with sensors 50A positioned along the perimeter edges of housing 12A. alternatively, sensors may be positioned on the opposite side of device 10 compared to secondary electrode 20. Such a configuration may be useful for EMG sensing to capture a larger area of signal detection while ECAP sensing will typically occur on the same face as a targeted nerve (e.g., the same side as secondary electrode 20).

In some embodiments, one or more of the sensors may be formed within the footer section of device 10. For example, FIG. 4C shows a side view of device 10 of housing 12B with sensors 50B positioned along the perimeter edge of footer 54 opposite of header 14. In this manner, the material forming footer 54 of device 10 may be used to electrically isolate sensors 50B from housing 12B and secondary electrode 12. Sensors 50B may be positioned in footer 54 on the same side as secondary electrode 20 or on other sides or perimeters of footer 54. Such a construction may help increase the separation distance between primary electrode 18 and sensors 50B as well as eliminate the need to modify the construction of housing 12B and make the assembly of device 10 more convenient. In some embodiments, one of primary electrode 18 or secondary electrode 20 may be arranged on footer 54.

In some embodiments, device 10 may include a plurality of sensors 50C arranged in a grid style fashion. FIG. 4D shows a side view of device 10 of housing 12C with sensors 50C arranged in two rows (i.e., an upper and lower row), each with multiple sensors 50C per row. A sensor 50C within each upper and lower row may form a sensor pair 56 configured to operate in the manner described above. By having two or more sensor pairs 56, the processing circuitry of device 10 may analyze the sensed signal acquired by each pair 56 to determine which pair 56 is best positioned for sensing the stimulation signal. The interrogation and analysis of sensors 50C may be performed periodically to determine if device migration has occurred with time. For example, during routine operation of device 10, the switching circuitry of device 10 may be configured to, at select intervals, switch between various sensor pairs 56 to determine which sensor pair is best positioned for sensory signal capture. The controller may then select the optimally placed sensor pair 56 to obtain sensory signals in response to stimulation therapy and use the obtained sensor information in the closed-loop feedback system to maintain optimization of the stimulation therapy parameters.

Additionally, or alternatively, by having plurality of sensors 50C within device 10, multiple sensory signals may be assessed at a given time. For example, one sensor pair 56 may be used to examine the ECAP of the target nerve or muscle tissue, while another set of sensors 50C may be used for EMG detection.

In another example, device 10 may include one or more sensors 50 positioned along the side opposite (not shown) of the side containing primary electrode 18 and secondary electrode 20. In such examples, sensors 50 will be facing away from the tibial nerve rather than facing towards the nerve. The sensors may be positioned along housing 12 and similar manner to that described above with respect to FIG. 4A. Additionally, or alternatively, such sensors 50 may be incorporated into header unit 14 or footer 54 using the techniques described above.

In yet another embodiment, device 10 may include an elongated sensor tail configured to be coupled to device 10 and coupled to the processing circuitry of device 10 to provide the closed loop functionality. FIG. 5 shows device 10A with footer 54A including a sensor tail 58 extending therefrom. Sensor tail 58 includes a plurality of sensors 60, substantially similar in terms of functionality to sensors 50. Sensor tail 58 allow for sensors 60 to be positioned further away from primary and secondary electrodes 18 and 20 to further reduce possible noise generated by the stimulation therapy. Sensor tail 58 modifies device 10A such that the device may no longer be considered “leadless,” but still operates in a leadless design for purposes of delivery of stimulation therapy. Thus, despite the presence of sensor tail 58, device 10A may still be considered a leadless neurostimulation device.

Referring now to implantation, FIG. 6A is a side view of a patient's leg 100 showing the leadless neurostimulation device 10 of FIG. 1 implanted, and FIG. 6B shows a cross-sectional schematic view of leadless neurostimulation device 10 implanted in leg 100 of a patient near the ankle adjacent to the tibial nerve 102. The cross section of leg 100 illustrates tibia 104, fibula 106, fibularis tertius 108, flexor digitorum longus 110, flexor hallucis longus 112, fibularis brevis 114, soleus 116, posterior tibial artery 118, posterior tibial vein 120, skin 122, cutaneous fat layer 124, and fascia layer 128. In some examples, device 10 may be implanted outside of fascia layer 128 near tibial nerve 102. Device 10 can be implanted through skin 122 and cutaneous fat layer 124 via a small incision 101 (e.g., about one to three cm) above the tibial nerve on a medial aspect of the patient's ankle. In other words, device 10 may be implanted in a pocket between skin and the fascia. Implantation outside fascia layer 128 may improve patient comfort and recovery because fascia layer 128 is not cut and does not need time to heal after implantation of device 10. While incision 101 is shown approximately horizontal to the length of the tibial nerve, other incisions or implantation techniques could be used according to physician preference.

Device 10 may be positioned adjacent to the region defined by flexor digitorum longus 110, flexor hallucis longus 112, and soleus 116 in which tibial nerve 102 is contained and implanted adjacent and proximal to fascia layer 128 with primary electrode 18 and secondary electrode 20 facing toward tibial nerve 102. Incision 101 preferably does not cross fascia layer 128 thereby reducing the risk of complications with the surgical procedure. In an embodiment, leadless neurostimulation device 10 may be implanted such that primary electrode 18 is oriented inferiorly relative to secondary electrode 20.

Optional testing of leadless neurostimulation device 10 may be performed to determine if device 10 has been properly positioned in proximity to tibial nerve 102 to elicit a desired response from an applied electrical stimulation. Such testing may be performed in conjunction with sensors 50 to ensure proper function of the device. In an example, device 10 may controlled by an external programmer to deliver test stimulation, and one or more indicative responses are monitored, such as toe flexion from simulation of the tibial motor neurons controlling the flexor hallucis brevis or flexor digitorum brevis, or a tingling sensation in the heel or sole of the foot excluding the medial arch. This can be associated with signal detection via sensor 50 to ensure proper functioning and establish a baseline. If such testing does not elicit appropriate motor or sensory responses, the practitioner may reposition device 10 and retest.

Once a practitioner has determined device 10 is properly positioned to provide an appropriate patient response to delivered stimulation therapy, housing 12 can be secured in place if needed. Such anchoring means may be optional as the natural shape of the region in which device 10 is implanted, and the shape of device 10 itself has shown good compatibility with the surrounding tissue thus preventing device 10 from shifting or rolling after implantation, in some embodiments, leadless neurostimulation device 10 may further include one or more suture points to help secure device 10 to fascia 102 or other parts of leg 100. In some embodiments, a suture anchor 130 may be included at the distal end of housing 12, opposite of the end attached to mounting plate 16.

The closed-loop protocol of device 10 may periodically or continually collect information from sensors 50 during routine operation of device 10 to assess proper placement and stimulation values. The controller can update the stimulation regimen as needed to maintain the delivery of proper stimulation therapy.

An advantage of the devices and methods described herein can be improved patient safety and satisfaction after implant. In contrast to other approaches, leadless neurostimulation device 10 does not require fascia layer 128 to be disturbed which may reduce risks affiliated with alternative procedures. Further, as device 10 is a unitary structure and can be hermetically sealed, the device is more robust than other lead-based stimulation units.

During operation, an electrical stimulation signal may be transmitted between primary electrode 18 and secondary electrode 20 through fascia layer 128. The electrical signal may be used to stimulate tibial nerve 102 which may be useful in the treatment of overactive bladder (OAB) symptoms of urinary urgency, retention, sexual dysfunction, urinary frequency and/or urge incontinence, or fecal incontinence. Sensors 50 (or others described above) may sense for the stimulation signal generated by primary and secondary electrodes 18 and 20, by measuring the EMG or ECAP of the surrounding nerve or muscle fibers. Based on the sensed information, collected in real time, the processing circuitry of device 10 may adjust the stimulation parameters of device 10 to optimize the stimulation therapy provided.

### EXAMPLES

### Example 1— Minimum Threshold Current

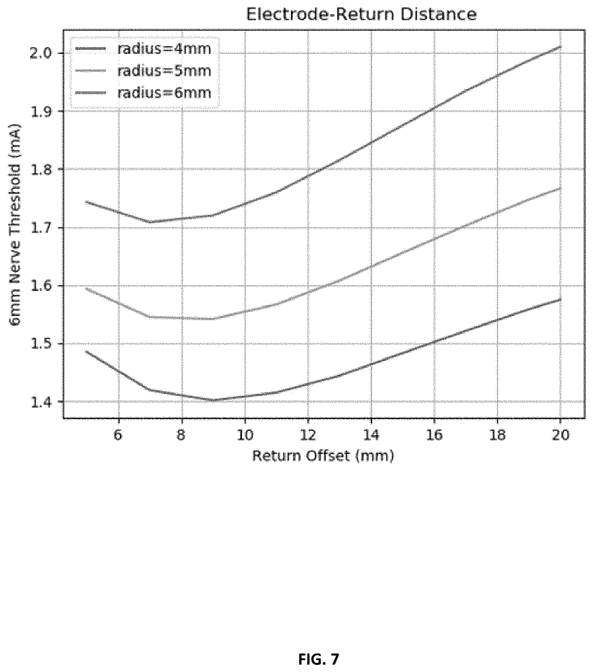

FIG. 7 is a plot showing examples of the minimum threshold level of electrical current needed to observe a tibial nerve response based on a function of the return offset (e.g., separation distance between primary electrode 18 and secondary electrode 20) in modeling studies. The studies also examined the minimal level of current needed to induce a simulated stimulation to a tibial nerve a select distance away as a function of secondary electrode size (e.g., circular radius). The minimum threshold was evaluated as the current required to stimulate a model of a single axon at the center of a tibial nerve (above the Y=0 axis) and a saphenous nerve (below the Y=0 axis) models.

Exemplary leadless neurostimulation devices were modeled based on the device of FIG. 1 and the power componentry of an InterStim Micro implantable system for Sacral Neuromodulation from Medtronic. The size of the contact surface of the primary electrodes was approximately 21.3 mm². The size (radius) and positioning of the secondary electrode was modified for the study. The leadless neurostimulation devices were placed in computer models approximately 0.5 mm from a simulated fascia layer with approximately 6 mm separation to the tibial nerve.

As shown in FIG. 7, the minimum threshold current needed to obverse stimulation response to the tibial nerve occurred within the range of about 6 mm to about 13 mm of a return offset for the tested radii. For a secondary electrode size of about 4 mm (50 mm²) a minimum threshold current of about 1.4 mA was observed at about a 9 mm offset. For a secondary electrode size of about 5 mm (79 mm²) a minimum threshold current of about 1.55 mA was observed at about 8 mm offset. For a secondary electrode size of about 6 mm (113 mm²) a minimum threshold current of about 1.7 mA was observed at about 7 mm offset. The smallest radii tested (4 mm) resulted in the lowest minimum threshold current (1.4 mA) but the largest return offset (9 mm).

### Example 2— Offset and Depth Comparison

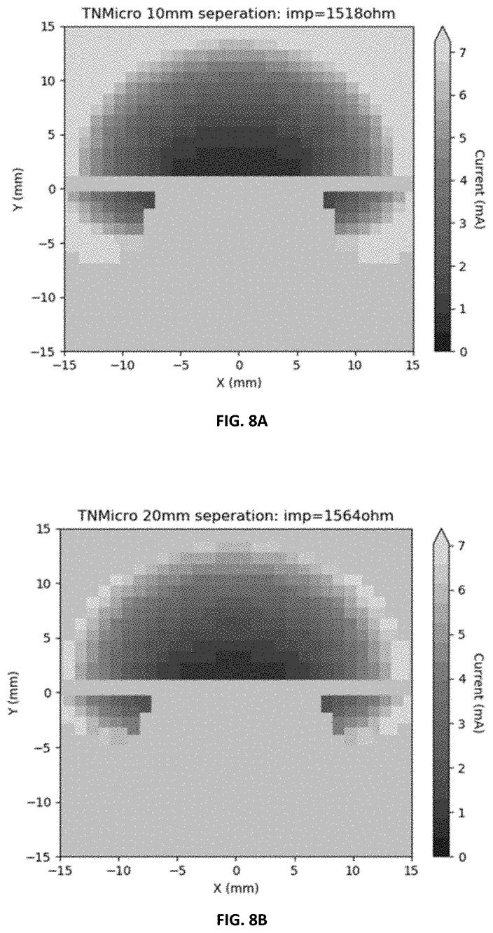

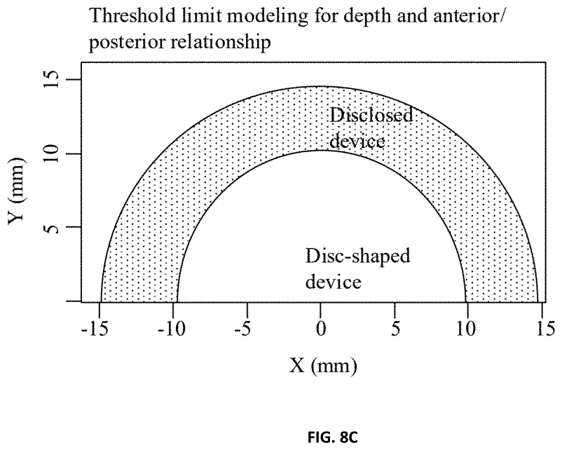

Simulations were conducted to examine the simulation depth as a function of the electrode offset (e.g., separation distance between primary and secondary electrodes) and stimulation voltage using modeling similar to Example 1. The size of the contact surface of the primary electrodes were approximately 21.3 mm² and the size of the secondary electrode was approximately 71 mm² (4.75 mm radius). The devices were tested at 10 mm and 20 mm electrode offsets. The leadless neurostimulation devices set in computer models approximately 0.5 mm from the fascia layer. FIGS. 8A and 8B are plots showing the threshold stimulation current in a cross-sectional view of the leg to capture the tibial nerve in the region above Y=0, and a cutaneous sensory nerve in the region below Y=0, for both 10 mm (FIG. 8A) and 20 mm (FIG. 8B) separation distances between the primary and secondary electrodes. The minimum threshold was evaluated as the current required to stimulate a model of a single axon at the center of a tibial nerve (above the Y=0 axis) and a saphenous nerve (below the Y=0 axis) models. The modeling demonstrated simulation obtainable within a radius of about 15 mm from the central axis of the device indicating that the disclosed device 12 may be useful in stimulating tibial nerves with deep or anterior tracks.

The modeling was compared to stimulation modeling for a disc-shaped stimulation device of 23 mm diameter and 2.2 mm thick. The disc stimulation device active electrode was modeled at about 12.5 mm² positioned at the center of the disc-shape and the return electrode was about 72.3 mm² and positioned at the perimeter edge of the side of the device. FIG. 8C is a plot showing modeling results FIG. 8C is a plot showing modeling results of stimulation threshold limit for the disclosed device compared to a disc-shaped stimulation device for depth and anterior/posterior relationship. The modeling demonstrated a notably reduced stimulation range (e.g., less than about 10 mm, e.g., 30% reduction in range) compared to the modeling of the present disclosed devices. It is believed that the reduction in operable range of the disc-shaped stimulation device may be due to the placement of the return electrode along the side of the device (e.g., not on the same side as the active electrode) as well as having the return electrode encircle the active electrode which negatively affect the possible pathway for the electrical stimulation. The modeling demonstrated that the disclosed device 12 may be useful in stimulating tibial nerves with deep or anterior tracks, particularly in comparison to disc-shaped stimulation devices.

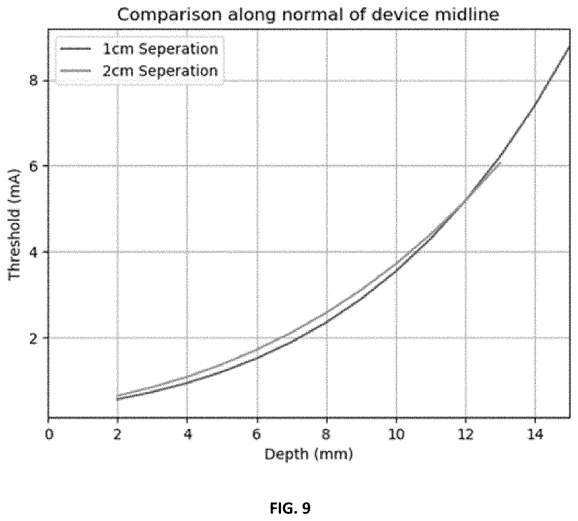

FIG. 9 shows the threshold current as a function of stimulation depth for both the 10 mm and 20 mm electrode offsets. The simulation depth was measured along the normal of the device midline. The results showed relatively similar results for both the 10 mm and 20 mm offset samples with slightly lower threshold values being determined for the 10 mm offset device at stimulation depths less than 12 mm.

### Example 3: Impedance and Depth Examination

The impedance and stimulation depth associated with the disclosed leadless neurostimulation devices were compared to a disc-shaped stimulation device using modeling similar to Example 1. The size of the contact surface of the primary electrode was approximately 21.3 mm² and the size of the secondary electrode was approximately 71 mm² (4.75 mm radius). The disc stimulation device was modeled to include a 23 mm diameter and 2.2 mm thick disc. The disc stimulation device active electrode was about 12.5 mm² positioned at the center of the coin-shape and the return electrode was about 72.3 mm² and positioned at the outer perimeter side of the device. Both devices were modeled approximately 0.5 mm from the fascia layer. The impedance of the disclosed leadless neurostimulation devices were found to be significantly lower than that of the disc stimulation device (e.g., modeled at about 1500 ohms or less compared to about 2100 ohms for the disc stimulation device). The comparatively lower impedance can allow for a higher current amplitude to be achieved for the same voltage, as well as better depth penetration. The comparatively lower impedance for the disclosed leadless electrodes may contributes to the device's ability to stimulate nerves over a larger area (laterally and depth) compared to the modeled disc-shaped device using comparable stimulation output.

Animal tests were also conducted to assess the practical impedance for representative neurostimulation devices of the disclose invention. Exemplary leadless neurostimulation devices were prepared by using an InterStim Micro implantable system for Sacral Neuromodulation from Medtronic that was modified to include the disclosed header unit 14 and secondary electrode 20. The device was implanted in ovine models approximately 0.5 mm from the fascia layer with approximately 6 mm separation to the tibial nerve. The observed impedance was surprisingly low at values of about 300 ohms. (e.g., approximately 316±130 ohms for the 10 mm separation and approximately 282±85 ohms for the 20 mm separation).

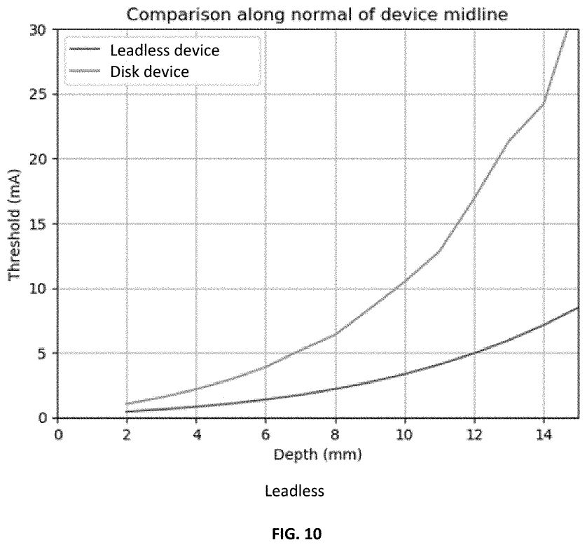

The threshold stimulation current as a function of stimulation depth was also modeled and compared between the disc stimulation device and the disclosed leadless neurostimulation device, which are plotted in FIG. 10. The disclosed leadless neurostimulation devices demonstrated a significant improvement in reducing the minimum threshold current to obtain tibial stimulation with increasing stimulation depth.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer). Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

1. A leadless neurostimulation device comprising:
a header unit comprising:
at least one primary electrode having a contact surface that defines an external surface of the leadless neurostimulation device; and
a housing comprising:
a secondary electrode positioned on the same side of the leadless neurostimulation device as the at least one primary electrode;
a footer coupled to the housing opposite of the header unit; and
a controller configured to operate in a closed-loop to:
transmit an electrical stimulation signal between the primary electrode to the secondary electrode to provide electrical stimulation therapy to a tibial nerve of a patient;
measure a physiologic parameter in response to transmission of the electrical stimulation therapy; and
adjust one or more parameters of the electrical stimulation signal based on the measured physiologic parameter.

2. The leadless neurostimulation device of claim 1, wherein the controller is configured to measure a physiologic parameter selected from an EMG signal, an ECAP signal, or both in response to the electrical stimulation signal.

3. The leadless neurostimulation device of claim 1, wherein the controller is configured to measure the physiologic parameter with one or more of the primary electrode or the secondary electrode.

4. The leadless neurostimulation device of claim 1, further comprising one or more sensors positioned along an exterior surface of the housing or the footer,
wherein the controller is configured to measure the physiologic parameter with the one or more sensors.

5. The leadless neurostimulation device of claim 1, wherein the controller is configured to measure the physiologic parameter with one or more of: the one or more sensors, the primary electrode, or the secondary electrode.

6. The leadless neurostimulation device of claim 4, wherein the one or more sensors comprise a plurality of sensors, wherein the controller is configured to transmit the electrical stimulation signal and measure the physiologic parameter at the same time.

7. The leadless neurostimulation device of claim 5, wherein at least two sensors of the plurality of sensors are aligned linearly with the primary electrode and the secondary electrode.

8. The leadless neurostimulation device of claim 5, wherein the plurality of sensors comprise a plurality or electrode pairs, each electrode pair aligned substantially parallel to along a length of the neurostimulation device.

9. The leadless neurostimulation device of claim 4, wherein at least one sensor of the one or more sensors is positioned along a surface of the housing, wherein the housing defines an aperture exposing the at least one sensor, the neurostimulation device further comprising a dielectric material set around the at least one sensor that electrically insulating the sensor from the housing.

10. The leadless neurostimulation device of claim 4, wherein at least one sensor of the one or more sensors is positioned within the footer, wherein the footer electrically insulates the at least one sensor from the housing.

11. The leadless neurostimulation device of claim 4, wherein at least one sensor of the one or more sensors is positioned along the perimeter edge of the neurostimulation device.

12. The leadless neurostimulation device of claim 4, wherein at least one sensor of the one or more sensors is positioned along a same side of the neurostimulation device as the secondary electrode.

13. The leadless neurostimulation device of claim 4, wherein each of the one or more sensors are separated from the primary electrode by an edge-to-edge separation distance of at least about 3 mm.

14. The leadless neurostimulation device of claim 4, further comprising a dielectric coating that at least partially encapsulates the leadless neurostimulation device, wherein a boundary defined by the dielectric coating defines the secondary electrode.

15. The leadless neurostimulation device of claim 1, further comprising a mounting plate, wherein the housing is coupled to a first side of the mounting plate and the header unit is coupled to a second side of the mounting plate

16. The leadless neurostimulation device of claim 1, wherein the device defines a total volume of about 1.5 cubic centimeters (cc) to about 3.5 cc.

17. A neurostimulation device comprising:
a header unit comprising:
at least one primary electrode having a contact surface that defines an external surface of the leadless neurostimulation device; and
a housing comprising:
a secondary electrode positioned on the same side of the leadless neurostimulation device as the at least one primary electrode;
a footer coupled to the housing opposite of the header unit;
a tail lead extending from the footer and comprising one or more sensors; and
a controller configured to operate in a closed-loop to:
transmit an electrical stimulation signal between the primary electrode to the secondary electrode to provide electrical stimulation therapy to a tibial nerve of a patient;
measure a physiologic parameter with the one or more sensors in response to transmission of the electrical stimulation therapy; and
adjust one or more parameters of the electrical stimulation signal based on the measured physiologic parameter.

18. The neurostimulation device of claim 16, wherein the controller is configured to transmit the electrical stimulation signal and measure the physiologic parameter at the same time.

19. The neurostimulation device of claim 16, wherein the header unit and the housing combined define a total volume of about 1.5 cubic centimeters to about 3.5 cubic centimeters.
